(19) United States
(12) Patent Application Publication
(10) Pub. No.: US 2026/0284205 A1
(43) Pub. Date: Sep. 24, 2026

(54) POLYIONIC COMPLEX OF NUCLEIC ACID AND CATIONIC POLYMER, SAID POLYIONIC COMPLEX HAVING POSITIVE SURFACE POTENTIAL AND BEING CAPABLE OF DELIVERING NUCLEIC ACID TO BRAIN TISSUE

(71) Applicant: The University of Tokyo, Tokyo (JP)

(72) Inventors: Kanjiro MIYATA, Tokyo (JP); Beob Soo KIM, Tokyo (JP); Mitsuru NAITO, Tokyo (JP)

(21) Appl. No.: 18/874,258

(22) PCT Filed: Jun. 12, 2023
(86) PCT No.: PCT/JP2023/021754

§ 371 Date: Mar. 12, 2025

(30) Foreign Application Priority Data
Jun. 13, 2022 (JP) 2022-094934

(51) Int. Cl. A61K 47/60

(52) U.S. Cl. CPC A61K 47/60

(57) ABSTRACT

The present invention provides a polyionic complex of a nucleic acid and a cationic polymer, the polyionic complex having a positive surface potential and being capable of delivering the nucleic acid to brain tissue. The polyionic complex of the present invention can be administered nasally, preferably to the nasal mucosa.

### TECHNICAL FIELD

The present invention relates to a polyionic complex of a nucleic acid and a cationic polymer, the polyionic complex having a positive surface potential and being capable of delivering the nucleic acid to brain tissue.

### BACKGROUND ART

Nucleic acid drugs are expected to be therapeutic agents for abnormal protein expression and activity since the drugs can regulate the protein expression. SiRNA is a short double-stranded RNA molecule that contains a sequence complementary to a target and silences target gene expression. Carriers containing siRNA have been proposed for administering siRNA to living organisms or delivering siRNA into cells (Non Patent Literature 1). Non Patent Literature 1 discloses a polyionic complex including siRNA and a cationic polymer. In addition, disclosed is a polyionic complex having a negative surface potential and including a short oligonucleotide and a cationic polymer (Non Patent Literature 2). Disclosed are a polyionic complex including a cationic polymer presenting a molecule for targeting the brain on the surface and an antisense oligonucleotide, and conditions of the cationic polymer necessary for presenting the molecule (Patent Literature 1). Disclosed is a polyionic complex including a nucleic acid and a cationic polymer with a characteristic side chain (Patent Literature 2).

### CITATION LIST

### Patent Literature

Patent Literature 1: WO 2019/240223A
Patent Literature 2: WO 2010/093036A

### Non Patent Literature

Non Patent Literature 1: J. Am. Chem. Soc., 2019, 141, 3699-3709
Non Patent Literature 2: Biomacromolecules, 2020, 21, 10, 4365-4376

### SUMMARY OF INVENTION

The present invention provides a polyionic complex of a nucleic acid and a cationic polymer, the polyionic complex having a positive surface potential and being capable of delivering the nucleic acid to brain tissue,

According to the present inventors, a polyionic complex of a nucleic acid and a cationic polymer, which has a positive surface potential, were able to effectively deliver the nucleic acid to the brain tissue, trigeminal nerve, and/or olfactory bulb by administration through the nasal mucosa (nasal mucosal administration). The present inventors were also able to effectively deliver molecules encapsulated in the polyionic complex (e.g., ribonuclease H (RNase H)) to the brain tissue, trigeminal nerve, and/or the olfactory bulb by administration through the nasal mucosa. The use of an antisense oligonucleotide (ASO) as a nucleic acid induced silencing of a target gene in a cell of brain tissue, indicating that the polyionic complex was excellent in delivering a nucleic acid into a cell. Encapsulation of RNase H in the polyionic complex that uses an ASO including a DNA region as a nucleic acid was also shown to allow more effective silencing of a target gene. The present invention is based on these findings.

The present invention provides the following:

[1] A polyionic complex comprising: (i) a block copolymer comprising (i-1) an uncharged hydrophilic polymer block and (i-2) a polymer block comprising a cationic amino acid; (ii) a nucleic acid (in particular, preferably a single-stranded nucleic acid that is an anionic polymer), wherein the polyionic complex has a positive surface potential (also referred to as “being positively charged”) and is capable of delivering the nucleic acid from outside a cell to cytoplasm.

[2] The polyionic complex according to [1], wherein an N/P ratio is 2.0 or more, where N denotes a valence of a positive charge in the block copolymer (e.g., the number of amino groups and guanidino groups in a peptide block), and P denotes a valence of a negative charge in the anionic polymer, for example, the nucleic acid (e.g., the number of phosphate groups).

[3] The polyionic complex according to [1], wherein the nucleic acid is an antisense oligonucleotide directed against a target RNA.

[4] The polyionic complex according to any one of [1] to [3], wherein the nucleic acid is an antisense oligonucleotide directed against a target RNA, the antisense oligonucleotide consisting of DNA or comprising at least a contiguous nucleotide portion consisting of DNA, and the polyionic complex encapsulates RNase H, whereby a hybrid of the target RNA and a DNA portion of an antisense oligo formed upon contact of the antisense oligonucleotide with the target RNA can be degraded.

[5] The polyionic complex according to any one of [1] to [4], wherein the uncharged hydrophilic polymer block is a polyalkylene glycol block (preferably polyethylene glycol).

[6] A composition comprising the polyionic complex according to any one of [1] to [4].

[7] The composition according to [6], which is nasally administered through nasal mucosa (nasal mucosal administration) of a subject.

[8] The composition according to [6] or preferably [7], for use in delivering the nucleic acid to brain tissue of a subject.

[9] The composition according to [6] or preferably [7], for use in delivering the nucleic acid to trigeminal nerve or olfactory bulb of a subject.

[10] The composition according to [6] or preferably [7], comprising antisense oligo DNA directed against a target gene, for use in suppressing expression of the target gene in brain tissue or trigeminal nerve cells of a subject.

[11] The composition according to any one of [8] to [10], preferably [10], which encapsulates RNase H.

[21] The polyionic complex as described above or a composition comprising the polyionic complex, wherein an average particle size according to dynamic light scattering is 30 to 150 nm.

[22] The polyionic complex as described above or a composition comprising the polyionic complex, particularly the polyionic complex according to [21] or a composition comprising the polyionic complex, wherein a polydispersity index is 0.3 or less, preferably 0.25 or less, and more preferably 0.2 or less.

[23] The polyionic complex as described above or a composition comprising the polyionic complex, particularly the polyionic complex according to [21] or [22] or a composition comprising the polyionic complex, wherein the polyionic complex is a hollow particle (i.e., a vesicle-type polyionic complex).

[24] The polyionic complex as described above or a composition comprising the polyionic complex, wherein the nucleic acid has a non-specific sequence.

[25] The polyionic complex as described above or a composition comprising the polyionic complex, wherein the nucleic acid has an inhibitory effect on a target RNA molecule.

[26] The polyionic complex as described above or a composition comprising the polyionic complex, wherein the nucleic acid comprises an antisense oligonucleotide, siRNA, shRNA, or miRNA directed against a target RNA molecule.

[27] The polyionic complex as described above or a composition comprising the polyionic complex, which encapsulates a substance, a molecule, a bioactive substance, or a contrast medium.

[28] The polyionic complex according to [24] or a composition comprising the polyionic complex, which encapsulates a substance, a molecule, a bioactive substance, or a contrast medium.

[29] The polyionic complex according to [25] or a composition comprising the polyionic complex, which encapsulates a substance, a molecule, a bioactive substance, or a contrast medium.

[30] The polyionic complex according to [26] or a composition comprising the polyionic complex, which encapsulates a substance, a molecule, a bioactive substance, or a contrast medium.

[31] The polyionic complex as described above or a composition comprising the polyionic complex, wherein the nucleic acid is an antisense oligonucleotide comprising at least a DNA region.

[32] The polyionic complex according to [31] or a composition comprising the polyionic complex, which encapsulates RNase H.

[33] The polyionic complex as described above or a composition comprising the polyionic complex, which has a positive surface potential and is administered through nasal mucosa.

[41]A composition for use in administering a nucleic acid, comprising the polyionic complex as described above.

[42] The polyionic complex according to [26] or a composition comprising the polyionic complex, for use in administering a nucleic acid.

[43] The polyionic complex according to [24] or a composition comprising the polyionic complex, for use in administering a substance, a molecule, a bioactive substance, or a contrast medium.

[44] The polyionic complex according to [25] or a composition comprising the polyionic complex, for use in administering a substance, a molecule, a bioactive substance, or a contrast medium.

[45] The polyionic complex according to [26] or a composition comprising the polyionic complex, for use in administering a substance, a molecule, a bioactive substance, or a contrast medium.

[46]A composition comprising the polyionic complex which is medically useful.

[47]A composition comprising the polyionic complex for use in treating cranial nerve disease.

[48] The composition according to [47], comprising a nucleic acid that targets and silences a gene responsible for the cranial nerve disease.

[49] The polyionic complex or a composition comprising the polyionic complex, which encapsulates a protein responsible for the cranial nerve disease, for protein replacement therapy for a deficiency or decrease in the protein.

[61] The composition according to any one of the above, wherein the subject has cranial nerve disease.

[63] The composition according to any one of the above, wherein the subject has cranial nerve disease in a cranial nerve region.

[63] The composition according to any one of the above, wherein the cranial nerve disease is a disease for which silencing by a nucleic acid provides a therapeutic benefit.

[63] The composition according to any one of the above, wherein the cranial nerve region is brain.

[64] The composition according to any one of the above, wherein the cranial nerve region is olfactory bulb.

[65] The composition according to any one of the above, wherein the cranial nerve region is trigeminal nerve.

[66] The composition according to any one of the above, wherein the cranial nerve region is brain.

### BRIEF DESCRIPTION OF DRAWINGS

FIG. 1 shows the formation of ASOsome (i.e., a vesicle-type polyionic complex containing an ASO) and the charged state thereof in an example of using an antisense oligonucleotide (ASO) as a nucleic acid and a block copolymer of polyethylene glycol (PEG) and a cationic polypeptide as a cationic polymer.

FIG. 2 shows the relationship between an N/P ratio and zeta potential of the resulting ASOsome, where the N/P ratio is the proportion (ratio) of the number of side-chain amino and guanidino groups responsible for positive charges to the number of phosphate and thiophosphate groups responsible for negative charges.

FIG. 3 shows the relationship between an average particle size (nm) according to dynamic light scattering and a polydispersity index (PDI) of the resulting ASOsome. The average particle size is indicated by a square mark, and the PDI is indicated by a diamond mark.

FIG. 4 shows the particle size distribution of the resulting positively or negatively charged ASOsome. The positively charged ASOsome is indicated by circular marks, and the negatively charged ASOsome is indicated by triangular marks.

FIG. 5 shows the distribution of positively or negatively charged ASOsome administered through the nasal mucosa in brain tissue.

FIG. 6 shows the distribution of positively or negatively charged ASOsome administered through the nasal mucosa in brain tissue. More specifically, FIG. 6 shows the accumulation (% dose) in each of the olfactory bulb, trigeminal nerve, and brain relative to the administered dose.

FIG. 7 shows accumulation in the olfactory bulb and trigeminal nerve of positively or negatively charged ASOsome administered through the nasal mucosa and a change in the accumulation over time (10, 30, 60, and 120 minutes after administration from the left).

FIG. 8 shows a change in the accumulation of positively or negatively charged ASOsome administered through the nasal mucosa over time in the olfactory bulb and trigeminal nerve. The ordinate indicates the amount of fluorescence (i.e., accumulation), and the abscissa indicates time (minutes).

FIG. 9 shows the accumulation (% dose) of ASOsome relative to the administered dose in each brain region.

FIG. 10 shows a graph of the amount and percentage of fluorescently labeled RNase H encapsulated in positively charged ASOsome as determined by fluorescence intensity measurement, results of fluorescence correlation spectroscopy showing that RNase H is encapsulated in positively charged ASOsome, and a transmission electron micrograph (TEM photograph) of ASOsome encapsulating RNase H.

FIG. 11 shows a knockdown effect of positively charged ASOsome administered through the nasal mucosa on a target gene in the brain (particularly in the cerebral cortex, striatum, and hippocampus). NT indicates no treatment, Naked ASO (unencapsulated free ASO) indicates naked ASO that is not encapsulated in a polyionic complex, and ASOsome+RNase H means a mixture of unencapsulated RNase H and ASOsome. The ordinate represents a value obtained by dividing the RNA content of the target gene Malat1 by the RNA content of the housekeeping gene Gapdh.

### DETAILED DESCRIPTION OF THE INVENTION

### Definitions

As used herein, the term “subject” refers to a vertebrate, which can be a mammal, for example, a primate including a human.

As used herein, the term “composition” refers to a mixture of one or more ingredients. The composition can contain, for example, a partial peptide and an aqueous solvent (e.g., water). The composition may further contain a pharmaceutically acceptable additive (e.g., an excipient, a carrier, etc.). A composition used in the treatment of a subject is referred to as a pharmaceutical composition.

As used herein, the term “treatment” encompasses prophylactic treatment and therapeutic treatment. The therapeutic treatment involves treating a subject with a disease or symptom, which can be given to retard or stop the worsening of symptoms or to prevent an increase in severity. The prophylactic treatment can be given to prevent the onset of a future disease or symptom.

As used herein, the term “effective amount” means the amount of an active ingredient that produces an expected pharmacological effect.

As used herein, the term “polyionic complex” means a polymer complex including a cationic polymer and an anionic polymer (e.g., a nucleic acid). In the complex, the cationic polymer and the anionic polymer form a bond by an ionic interaction. In particular, a complex formed by a polymer complex of a copolymer of an uncharged hydrophilic polymer block (e.g., polyethylene glycol and polyoxazoline, for example, polyethylene glycol) and a cationic polymer block with a nucleic acid can form a micelle or hollow particle, the surface of which is considered to be coated with a uncharged hydrophilic polymer block (e.g., polyethylene glycol and polyoxazoline). The term “uncharged hydrophilic polymer block” means a polymer block that is uncharged and hydrophilic in saline at room temperature and in blood in the body. The term “uncharged” means nonionic. A polyionic complex can be formed of a nucleic acid and a copolymer of an uncharged hydrophilic polymer block and a cationic polymer block.

As used herein, the term “block copolymer” refers to a polymer in which two or more different polymers are linked in a monomeric unit at the edges, and therefore, the block copolymer contains two or more blocks each containing a polymer. When a polymer having physiochemically different properties is loaded on each block, the block copolymer can combine the different properties, and thereby the polymer can be made highly functional. For example, a block copolymer of an uncharged hydrophilic polymer block and a cationic polymer block acquires biocompatibility due to the nature of the uncharged hydrophilic polymer block, in addition to being able to form a complex with an anionic polymer through an ionic interaction.

As used herein, the term “mRNA” means messenger RNA. mRNA, known as a nucleic acid encoding a protein, may be translated to the protein in the cytoplasm. Gene knockdown (i.e., suppression of expression) can be achieved by targeting mRNA. For example, an antisense oligonucleotide, siRNA, shRNA, and microRNA directed against mRNA are tools for knocking down gene expression of mRNA as a target.

As used herein, the term “antisense oligonucleotide” may refer to a nucleic acid such as DNA, RNA, a hybrid of DNA and RNA, or a modified nucleic acid (including a gapmer and a mixmer). An antisense oligonucleotide targeting RNA preferably includes DNA to induce recognition and degradation by RNase H, and more preferably is a molecule containing DNA or a molecule (i.e., a gapmer) having a modified nucleic acid (e.g., LNA) at each end of the DNA molecule. The RNase H acts as an endonuclease against a hybrid molecule of RNA and DNA, thereby degrading the target RNA and inducing silencing. The antisense oligonucleotide targeting RNA is preferably a modified nucleic acid from the viewpoint of stability and/or strength of binding to the RNA. The antisense oligonucleotide can be used for silencing the expression of target RNA as well as exon skipping to remove specific exons of mRNA (e.g., an antisense oligonucleotide to induce exon skipping for dystrophin has been developed). The antisense oligonucleotide usually has a length of about 14 to about 35 mers (e.g., about 20 to about 30 mers). The siRNA usually has a length of about 20 to about 25 mers (e.g., about 21 to about 23 mers). The shRNA usually has a rod and a loop of about 20 to about 25 base pairs (e.g., about 21 to about 23 base pairs). The miRNA usually, for example, has a length of about 20 to about 25 mers and a sequence complementary to the target RNA to suppress the expression. The antisense oligonucleotide, siRNA, shRNA, and miRNA can preferably suppress the target RNA without inducing excessive or pharmaceutically unacceptable inflammation.

Examples of the modified nucleic acid include a nucleic acid modified with a fluorescent dye, a biotinylated nucleic acid, and a nucleic acid into which a cholesteryl group is introduced. The RNA may have 2′-O-methyl or 2′-fluoro or 2′-methoxyethyl (MOE) modifications to the bases in order to increase stability, and a phosphodiester linkage of the nucleic acid backbone may be replaced by a phosphorothioate linkage. Examples of artificial nucleic acids include nucleic acids in which an oxygen atom at the 2′ position and a carbon atom at the 4′ position are cross-linked. Examples of the artificial nucleic acid include a locked nucleic acid (LNA), cross-linked DNA in which an oxygen atom at position 2′ and a carbon atom at position 4′ are cross-linked via methylene; ENA, in which an oxygen atom at position 2′ and a carbon atom at position 4′ are cross-linked via ethylene; a cross-linked nucleic acid (BNA) such as BNA^{COC}, in which an oxygen atom at position 2′ and a carbon atom at position 4′ are cross-linked via —CH₂OCH₂—, and BNA^{NC}, in which an oxygen atom at position 2′ and a carbon atom at position 4′ are cross-linked via —NR—CH₂— (where R is a methyl or hydrogen atom); cMOE, in which an oxygen atom at position 2′ and a carbon atom at position 4′ are cross-linked via —CH₂(OCH₃)—; cEt, in which an oxygen atom at position 2′ and a carbon atom at position 4′ are cross-linked via —CH₂(CH₃)—; AmNA, in which carbon atoms at positions 2′ and 4′ are cross-linked via an amide; scpBNA, in which an oxygen atom at position 2′ and a carbon atom at position 4′ are cross-linked via methylene to form a cyclopropane at position 6′; and a peptide nucleic acid (PNA), in which a polymer of amide-linked N-(2-aminoethyl)glycine, instead of deoxyribose or ribose, is the backbone. Examples of the RNA include artificial RNA for gene silencing such as siRNA and shRNA, microRNA (miRNA), non-coding RNA such as an aptamer, and natural RNA such as mRNA. Such RNA can be modified to be stabilized in vivo.

### <Polyionic Complex of Invention>

The present invention provides a polyionic complex including: (i) a block copolymer including (i-1) an uncharged hydrophilic polymer block and (i-2) a cationic polymer block (polymer block containing a cationic amino acid); (ii) a nucleic acid. The present invention also provides a polyionic complex including: (i) a block copolymer including (i-1) an uncharged hydrophilic polymer block and (i-2) a cationic polymer block (polymer block containing a cationic amino acid); (ii) a nucleic acid, wherein the polyionic complex has a positive surface potential (also referred to as “being positively charged”). The present invention further provides a polyionic complex including: (i) a block copolymer including (i-1) an uncharged hydrophilic polymer block and (i-2) a cationic polymer block (polymer block containing a cationic amino acid); (ii) a nucleic acid, wherein the polyionic complex has a positive surface potential and is capable of delivering the nucleic acid from outside a cell to cytoplasm.

The uncharged hydrophilic polymer block has no overall charge and is hydrophilic. The uncharged hydrophilic polymer block contributes to the formation of the polyionic complex and is suitable for imparting biocompatibility to the polyionic complex. Examples of the uncharged hydrophilic polymer blocks include a block of polyalkylene glycol, for example, a block of polyethylene glycol. Examples of the uncharged hydrophilic polymer block also include polyoxazoline.

The uncharged hydrophilic polymer block can have, for example, a number average degree of polymerization of 20 to 300, for example, 30 to 100, for example, 40 to 60. The uncharged hydrophilic polymer block may have, for example, a number-average molecular weight of 1 kDa to 20 kDa, for example, 1.5 kDa to 15 kDa, for example, 2 kDa to 12 kDa.

According to the invention, the cationic polymer may be a copolymer with an uncharged hydrophilic polymer block. The cationic polymer block is a polymer block having an overall positive charge and usually substantially free or completely free of negatively charged monomeric units. The cationic polymer block may be a polymer having a natural cationic amino acid (e.g., lysine and ornithine) as a monomeric unit. The cationic polymer block may also be a polymer having a non-natural cationic amino acid as a monomeric unit. The cationic polymer block may be a polymer having a natural cationic amino acid and a non-natural cationic amino acid as monomeric units. Examples of the non-natural cationic amino acid are disclosed in WO 2010/093036A. Examples of the non-natural cationic amino acid include an amino acid in which a group selected from the group consisting of the following formulas:

—NH—(CH₂)ₚ₁—[NH—(CH₂)_{q1}-]ᵣ₁—NH₂ (i);

—NH—(CH₂)ₚ₂—N[—(CH₂)_{q2}—NH₂]₂ (ii);

—NH—(CH₂)ₚ₃—N{[—(CH₂)_{q3}—NH₂][—(CH₂)_{q4}—NH]ᵣ₂H} (iii);

and —NH—(CH₂)ₚ₄—N{—(CH₂)_{q5}—N[—(CH₂)_{q6}—NH₂]₂}₂ (iv),

where p1 to p4, q1 to q6, and r1 to r2 are each independently of each other and consist of groups that are integers of 1 to 5, is directly or indirectly linked via a peptide bond to carboxy group of the side chain of glutamic acid or aspartic acid. In one aspect, p1 and q1 may be 2, and r1 may be any natural number from 1 to 5, preferably 1, 2, or 3. The non-natural amino acid may be, for example, a molecule in which a cationic side chain (e.g., NH₂—(CH₂)ₙ—NH₂ or NH₂—(CH₂)ₙ—NH—C[(═NH)(—NH₂)]) is linked directly or indirectly via a peptide bond to a glutamic acid or aspartic acid side chain, where n can independently be a natural number from 1 to 8, e.g., 1, 2, 3, 4, 5, 6, 7, or 8. The number average degree of polymerization of the cationic polymer block may be, for example, 30 to 100, 40 to 80, or 50 to 70.

For example, (i) a block copolymers including (i-1) an uncharged hydrophilic polymer block and (i-2) a cationic polymer block may further contain a hydrophobic polymer block. In this aspect, the block copolymer may preferably contain an uncharged hydrophilic polymer block, a cationic polymer block, and a hydrophobic polymer block in this order. In addition, for example, the cationic polymer block (i-2) may include a hydrophobic monomeric unit in addition to the cationic monomeric unit. In this aspect, the cationic polymer block may be a copolymer (e.g., a statistical copolymer) including a cationic monomeric unit and a hydrophobic monomeric unit (see Kim et al., ACS Cent. Sci., 5, 1866-1875, 2019). The monomeric unit of the hydrophobic polymer block or the hydrophobic monomeric unit is not limited but may have, for example, a structure in which a hydrophobic group is linked to a carboxyl group of a side chain of glutamic acid or aspartic acid via a peptide bond. The hydrophobic group may be substituted or unsubstituted alkyl, substituted or unsubstituted alkynyl, substituted or unsubstituted alkenyl, substituted or unsubstituted cycloalkyl, substituted or unsubstituted cycloalkynyl, substituted or unsubstituted cycloalkenyl, or substituted or unsubstituted aryl, and the substituent may be a hydrophobic group. Examples of the hydrophobic group include substituted or unsubstituted alkane, substituted or unsubstituted alkyne, substituted or unsubstituted alkene, substituted or unsubstituted cycloalkyl, and substituted or unsubstituted aryl. In one aspect, alkyl can be lower alkyl, alkynyl can be lower alkynyl, and alkenyl can be lower alkenyl. The lower alkyl, lower alkynyl, and lower alkenyl mean molecules having 1 to 6 carbon atoms and include molecules having 1 to 4, 1 to 3, or 1 to 2 carbon atoms. In one aspect, the hydrophobic group can be —(CH₂)ₙ₂—H, where n2 can be any natural number from 4 to 15, for example, 4, 5, 6, 7, 8, 9, or 10, and in one aspect, the hydrophobic group can be an aromatic alkyl group, wherein the alkyl group can be an alkyl group having 1 to 4 carbon atoms. The hydrophobic polymer block may be introduced for stabilization of the polyionic complex. The polyionic complex is described in detail in Hori et al., Biomacromolecules, 19, 4113-4121, 2018, Chuanoi et al., Polymer Journal, 46, 130-135, 2014, Chuanoi et al., Biomacromolecules, 15, 2389-2397, 2014, Uchida et al., J. Am. Chem. Soc., 136, 12396-12405, 2014, Kim et al., ACS Cent. Sci., 5, 1866-1875, 2019, and Yum et al., Journal of Controlled Release, 342, 148-156, 2022, which are incorporated herein by reference in their entirety.

When (i) a block copolymer including (i-1) an uncharged hydrophilic polymer block and (i-2) a cationic polymer block (polymer block containing a cationic amino acid) and (ii) a nucleic acid are mixed together in an aqueous solution, the above (i) and (ii) form a complex to form a polyionic complex. The polyionic complex may be preferably in the form of a micelle or a hollow particle (vesicle), and more preferably in the form of a vesicle. The micelle and the hollow particle can be easily formed individually by adjusting, for example, the length of the uncharged hydrophilic polymer block, although the formation method is not limited.

The polyionic complex may have, for example, an average particle size according to dynamic light scattering (DLS) of 30 to 150 nm, for example, 50 to 140 nm, for example, 60 to 130 nm, for example, 70 to 120 nm, for example, 80 to 110 nm, for example, 90 to 110 nm. The polydispersity index of the polyionic complex may be, for example, 0.3 or less, for example, 0.25 or less, for example, 0.2 or less.

The polyionic complex may have a positive surface potential or a negative surface potential, but preferably has a positive surface potential. To obtain a polyionic complex having a positive surface potential, the N/P ratio can be equal to or greater than the first predetermined proportion. The first predetermined proportion can be, for example, more than 1.8, preferably 2.0 or more, more preferably 2.1 or more, and even more preferably 2.2 or more, for example, 2.3 or more, 2.4 or more, 2.5 or more, 2.6 or more, or 2.7 or more. In this manner, the surface potential of the polyionic complex can be modulated so that it is positive, and the N/P ratio can be equal to or smaller than the second predetermined proportion in order to obtain a polyionic complex having a negative surface potential. The second predetermined proportion can be, for example, less than 1.8, preferably 1.7 or less, more preferably 1.6 or less, and even more preferably 1.5 or less, 1.4 or less, 1.3 or less, 1.2 or less, or 1.1 or less. In this manner, the surface potential of the polyionic complex can be modulated so that it is negative, where N denotes a valence of the positive charge in the block copolymer (e.g., the number of amino and guanidino groups in the peptide block), and P denotes a valence of the negative charge in the anionic polymer, e.g., the nucleic acid (e.g., the number of phosphate groups). In the case of using a nucleic acid that does not have phosphorus (P), those skilled in the art would be able to adjust the polyionic complex to have a positive surface potential or a negative surface potential, as appropriate. The surface potential of the polyionic complex can be evaluated, for example, by the zeta potential thereof. The zeta potential can be measured using, for example, electrophoretic light scattering. The surface potential can be, for example, the surface potential in a buffer solution (e.g., PH 7.4 in 10 mM Hepes buffer). In one preferred aspect, the N/P ratio is greater than or equal to a first predetermined value, and the first predetermined value may be 2.0 or more, more preferably 2.1 or more, and even more preferably 2.2 or more.

According to the present invention, the nucleic acid may preferably be an anionic polymer. On the other hand, a nucleic acid that is not anionic, such as morpholino oligo and a peptide nucleic acid, can also be used as a nucleic acid in the present invention, for example, by forming a block copolymer with an anionic polymer or by mixing with anionic polymer. The nucleic acid that is an anionic polymer can be, or contain, for example, one or more nucleic acids selected from the group consisting of single-stranded DNA, single-stranded RNA, double-stranded DNA, double-stranded RNA, single-stranded modified nucleic acid, and a double-stranded modified nucleic acid. The nucleic acid that is an anionic polymer can be a polymer containing a monomeric unit selected from the group consisting of (α) DNA and RNA, (β) DNA and modified nucleic acid, (γ) RNA and modified nucleic acid, and (δ) DNA, RNA and modified nucleic acid. In a preferred aspect, a highly stable nucleic acid is linked to one end, and more preferably both ends, of a less stable nucleic acid.

For example, the nucleic acid that is an anionic polymer may be linked in the order of DNA block-RNA block-DNA block from the 5′ end, with each block containing at least one or more, two or more, three or more, or four or more nucleic acid monomeric units.

For example, the nucleic acid that is an anionic polymer may be linked in the order of modified nucleic acid block-RNA block-modified nucleic acid from the 5′ end, with each block containing at least one or more, two or more, three or more, or four or more nucleic acid monomeric units.

For example, the nucleic acid that is an anionic polymer may be linked in the order of modified nucleic acid block-DNA block-modified nucleic acid from the 5′ end, with each block containing at least one or more, two or more, three or more, or four or more nucleic acid monomeric units. In the above, the symbol “-” means a linkage between monomeric units, which may include a direct linkage and an indirect linkage through a linker. In a preferred aspect, the modified nucleic acid block can be a block including LNA as a monomeric unit.

When a nucleic acid with high stability as described above is linked to one end, more preferably both ends, of a nucleic acid with low stability, the nucleic acid can be stabilized in vivo or in a cell to maintain its function or effect.

In the present invention, the target gene that can be inhibited by the nucleic acid (e.g., by delivery of RNAi, microRNA, or antisense oligonucleotide) is not limited but may be a gene that can receive a therapeutic or prophylactic benefit for a brain nervous system disease upon knockdown. The disease to be treated may be a brain nervous system disease for which knockdown of the gene may provide a therapeutic or prophylactic benefit. For example, in the treatment of amyotrophic lateral sclerosis (ALS), a nucleic acid such as antisense oligonucleotide directed against SOD1 (Cu/Zn superoxide dismutase), FUS (fused in sarcoma), C90RF72, or ATXN2 (ataxin2) may be administered to a subject in need thereof. Also, in the treatment of Alzheimer's disease, a nucleic acid such as an antisense oligonucleotide directed against tau may be administered to a subject in need thereof. In the treatment of Parkinson's disease, a nucleic acid such as an antisense oligonucleotide directed against LRRK2 (leucine-rich repeat kinase 2), SNCA (α-synuclein), HTT (huntingtin), or SNP2 (SNP rs362331) may be administered to a subject in need thereof.

The polyionic complex of the present invention may or may not have crosslinks between the polymers. In a preferred aspect, the polyionic complex may have a crosslink between the block copolymers. In a preferred aspect, there are no crosslinks between the nucleic acids or between the nucleic acid and the block copolymer. In a preferred aspect, the polyionic complex has no crosslinks between the polymers.

The polyionic complex of the present invention is suitable for delivering a nucleic acid from the outside of a cell to the inside of the cell. The polyionic complex of the present invention is also, in one aspect, suitable for delivery to the nervous system, including brain tissue, by nasal administration through the nasal mucosa. The polyionic complex of the present invention need not include a targeting molecule for a specific cell. For example, WO 2019/240223A discloses a polyionic complex micelle that expresses a GLUT1 ligand (i.e., a molecule with binding properties for GLUT1) on the surface, and the micelle is intended to penetrate the brain-blood barrier (BBB) by affinity for GLUT1. However, the present invention does not require such a targeting molecule.

According to the present invention, the nucleic acid may be an antisense oligonucleotide. The antisense oligonucleotide has a sequence capable of hybridizing to a target RNA in an intracellular environment (preferably a complementary sequence) and acts by hybridizing to the target RNA. The sequence capable of hybridizing to a target RNA in the intracellular environment can be appropriately designed by those skilled in the art, and such an antisense oligonucleotide can be obtained. The antisense oligonucleotide usually has a length of about 14 to about 35 mers. The antisense oligonucleotide can be a modified nucleic acid (e.g., LNA, morpholino oligonucleotide, etc.). The antisense oligonucleotide can be DNA. The antisense oligonucleotide may have a structure in which modified nucleic acids are linked to both ends of DNA. According to the present invention, the nucleic acid may be any other nucleic acid. The nucleic acid is as described in detail above. The antisense oligonucleotide may be one that can hybridize to the target RNA in the intracellular environment. Hybridization means formation of perfect Watson-Crick base pairs to complementary sequences and formation of an overall stable hybrid due to partial imperfect Watson-Crick base pairs in other portions (e.g., including hybridization to a target in exon skipping). Hybridization can be specific or selective for the target RNA.

As will be described later, when RNase H is encapsulated in a polyionic complex, the antisense oligonucleotide preferably has a DNA portion (DNA block) because when the DNA portion hybridizes with the target RNA, RNase H enzymatically degrades the DNA-RNA hybrid portion, thereby increases an inhibitory effect of the antisense oligonucleotide on the target RNA.

A substance, for example, a bioactive substance, such as RNase H, may be included in the composition at a concentration of, but not limited to, 0.01 mg/mL or more, 0.02 mg/mL or more, 0.03 mg/mL or more, 0.04 mg/mL or more, 0.05 mg/mL or more, 0.06 mg/mL or more, 0.07 mg/ml or more, 0.08 mg/ml or more, 0.09 mg/ml or more, 0.10 mg/mL or more, 0.15 mg/mL or more, 0.2 mg/mL or more, or 0.3 mg/mL or more. In a particularly preferred aspect, a substance, for example, a bioactive substance, such as, RNase H, can be encapsulated in the polyionic complex and contained in the composition at the concentration described above. Encapsulation of a substance, for example, a bioactive substance, such as, RNase H, in the polyionic complex can be performed by mixing the polyionic complex with a substance, for example, a bioactive substance, such as, RNase H. Mixing can be performed by, for example, vigorous stirring, stirring using a vortex mixer, or the like.

The bioactive substance to be encapsulated can be produced using various cells. When the bioactive substance is a protein, the protein can be produced with various cells by a method well known to those skilled in the art, and the protein can be recovered. The recovered protein can be encapsulated in the polyionic complex after being purified (e.g., purification by gel filtration, affinity purification, etc.) as necessary.

According to the present invention, the polyionic complex is, for example, a vesicle-type polyionic complex, and a substance may be encapsulated therein. As the substance, a bioactive substance or a contrast medium for imaging can be used. The bioactive substance may be a substance that has a benefit when delivered to the brain or nerves, and examples thereof include a low molecular weight compound, a protein, an antibody, and an antigen-binding fragment thereof. Examples of the contrast medium for imaging include a contrast medium for nuclear magnetic resonance imaging (MRI) and a contrast medium for positron emission tomography (PET). Examples of the low molecular weight compound include a therapeutic agent and prophylactic agent for cranial nerve disease. Examples of the protein include a protein expressed in brain cells, a proteolytic enzyme, and a nucleolytic enzyme (e.g., RNase H). It is understood that the nucleic acid need not encode meaningful information if the sole purpose is to deliver the encapsulated substance to the target tissue or cell. In this case, the nucleic acid is a polymer having excellent biocompatibility and exhibits technical significance only in the structure formation of the polyionic complex.

According to Examples described later, the polyionic complex can deliver a nucleic acid to the cytoplasm at least from outside a cell. According to Examples described later, the polyionic complex can deliver a nucleic acid to brain nerves including the brain, olfactory bulb, and trigeminal nerve by nasal administration through the nasal mucosa. The delivered nucleic acid may also be delivered to the cytoplasm of the cell of the nerve. The delivery efficiency can be enhanced by the polyionic complex being positively charged. In addition, when the nucleic acid is an antisense oligonucleotide containing DNA, suppression (particularly, silencing) of RNA can be effectively induced in a cell, and the polyionic complex containing RNase H can more effectively induce suppression (particularly, silencing or degradation) of RNA.

According to the present invention, the polyionic complex further contains a molecule that promotes cell membrane permeability, such as a cell membrane-penetrating peptide (CPP), and may express the molecule on the surface or may not contain or express the molecule. According to the present invention, the polyionic complex can deliver a nucleic acid to the brain with favorable efficiency without using such a molecule.

### <Composition of the Invention>

The present invention provides a composition containing a polyionic complex. The composition can be a pharmaceutical composition. The composition or pharmaceutical composition may further contain a pharmaceutically acceptable additive (such as an excipient or a carrier) in addition to the polyionic complex.

According to the present invention, the composition containing a polyionic complex can be nasally administered to a subject. In particular, the composition of the present invention can be nasally administered to the nasal mucosa (also referred to as “nasal mucosal administration”), whereby the nucleic acid contained in the polyionic complex can be delivered to any or all of brain tissue, trigeminal nerve, and olfactory bulb as a target tissue. In one aspect, the polyionic complex is positively charged, and preferably, the target tissue can be brain tissue and/or trigeminal nerve. In one aspect, the polyionic complex is negatively charged, and preferably, the target tissue can be trigeminal nerve. After being delivered to a target tissue, the polyionic complex of the present invention can be taken up into a cell to deliver a nucleic acid to the cytoplasm. Hence, the polyionic complex can be advantageously used to control gene expression (particularly gene expression suppression, or knockdown, etc.) using a nucleic acid in a target tissue. In particular, when the nucleic acid is an antisense oligonucleotide containing DNA, suppression (particularly, silencing) of RNA can be effectively induced in a cell, and the polyionic complex containing RNase H can more effectively induce suppression (particularly, silencing or degradation) of RNA. From the viewpoint of encapsulating a substance, the polyionic complex of the present invention can be preferably a vesicle-type polyionic complex. The subject may, in one aspect, have a cranial nerve disease, Examples of the cranial nerve disease are listed below. When the active ingredient is a nucleic acid, the cranial nerve disease may be, for example, a disease in which silencing by the nucleic acid provides a benefit. In one aspect, the subject may have a disease in the cranial nerve region. The cranial nerve region may be selected from the group consisting of, for example, the brain, olfactory bulb, and trigeminal nerve,

According to the present invention, a composition including the polyionic complex can be a nasal administration formulation for nasal mucosa. In the most preferred aspect, in a composition containing the polyionic complex, the polyionic complex has a positive surface potential, and the composition is nasally administered to the nasal mucosa.

In one aspect, the composition, pharmaceutical composition, and nasal administration formulation (formulation for nasal mucosal administration) of the present invention may be used to treat a neurological disease (cranial nerve disease), such as a disease of the brain or central nervous system (CNS). Examples of the typical disease include Alzheimer's disease, Parkinson's disease, amyotrophic lateral sclerosis, frontotemporal dementia, vascular dementia, dementia with Lewy bodies, Pick's disease, primary age-related tauopathy, and progressive supranuclear palsy. In some embodiments, the disease can be a tauopathy, a prion disease (e.g., bovine spongiform encephalopathy, scrapie, Creutzfeldt-Jakob disease, kuru, Gerstmann-Straussler-Scheinker disease, chronic wasting disease, and fatal familial insomnia, etc.), bulbar palsy, a motor neuron disease, or a nervous system heterodegenerative disease (e.g., Canavan's disease, Huntington's disease, neuroceroid lipofuscinosis, Alexander's disease, Tourette's syndrome, Menke's frizzy hair syndrome, Cockayne's syndrome, Hallervorden-Spatz syndrome, Lafora disease, Rett's syndrome, hepatolenticular degeneration, Lesch-Nyhan syndrome, Friedreich's ataxia, spinal muscular atrophy, and Unferlicht-Lundborg syndrome, etc.). In some embodiments, the disease is stroke or multiple sclerosis. In some embodiments, the patient may be asymptomatic, but has a marker associated with a disease of the brain or CNS. In this aspect, mRNA where silencing of gene provides therapeutic significance in the above disease can be targeted. In this case, the polyionic complex of the present invention would also encapsulate a therapeutic agent for one of these diseases.

In one aspect, the composition, pharmaceutical composition, and nasal administration formulation (formulation for nasal mucosal administration) of the present invention can be used in the treatment of brain tumors, such as glioma, glioblastomas multiforme, meningioma, astrocytoma, acoustic neuroma, chondroma, oligodendroglioma, medulloblastoma, ganglioglioma, schwannoma, neurofibroma, neuroblastoma, and an epidural, intramedullary, or intradural tumor. In this aspect, mRNA where silencing of gene provides therapeutic significance in the above disease can be targeted. In this case, the polyionic complex of the present invention would also encapsulate a therapeutic agent for one of the brain tumors.

The composition, pharmaceutical composition, and nasal administration formulation of the present invention may further contain a pharmaceutically acceptable additive. The pharmaceutically acceptable additive may be, for example, a pharmacologically or physiologically acceptable ingredient, such as a disintegration aid, an antioxidant or an oxidation inhibitor, a stabilizer, an antiseptic or a preservative, a bactericide or an antibacterial agent, an antistatic agent, a flavoring substance or a masking agent, a colorant, a deodorant or a perfume, a refreshing agent, and an antifoaming agent.

These other ingredients can be used singly or in combinations of two or more.

### <Other Inventions>

The present invention provides a method of administering a nucleic acid to a subject in need thereof, the method including administering to the subject an effective amount of the polyionic complex. Administration can be performed by nasal administration to the nasal mucosa. The polyionic complex may be delivered to the cranial nerve region. In one aspect, the cranial nerve region may be selected from the group consisting of the brain, olfactory bulb, and trigeminal nerve.

The present invention provides a method of administering a substance to a subject in need thereof, the method including administering to the subject an effective amount of the polyionic complex encapsulating the substance. Administration can be performed by nasal administration to the nasal mucosa. The polyionic complex may be delivered to the cranial nerve region. In one aspect, the cranial nerve region may be selected from the group consisting of the brain, olfactory bulb, and trigeminal nerve.

The present invention provides the use of the polyionic complex in the manufacture of a composition (or a drug) for use in delivering a nucleic acid to a cranial nerve region selected from the group consisting of the brain, olfactory bulb, and trigeminal nerve.

The present invention provides the use of the polyionic complex encapsulating the substance in the manufacture of a composition (or a drug) for use in delivering the substance to a cranial nerve region selected from the group consisting of the brain, olfactory bulb, and trigeminal nerve.

The present invention provides a composition or a pharmaceutical composition to be used for the above method.

All of the composition, pharmaceutical composition, and drug can be preferably administered nasally through the nasal mucosa. Thus, the composition, pharmaceutical composition, and drug may have a composition suitable for nasal administration formulations.

The composition, pharmaceutical composition, and drug of the present invention can deliver a nucleic acid or an encapsulating substance to lung tissue,

The pharmaceutical composition of the present invention may have medical utility. For example, the pharmaceutical compositions of the present invention may be used in the treatment of cranial nerve disease. Examples of the cranial nerve disease include a psychotic disorder, a depressive disorder, a mood disorder, anxiety, a sleep disorder, a dementia, and a substance-related disorder. Examples of the dementia include, but are not limited to, Alzheimer's disease and Creutzfeldt-Jakob disease. Examples of the substance to be encapsulated include an active pharmaceutical ingredient for each of the disease. The polyionic complex of the present invention may contain a nucleic acid that targets (particularly, silences or knocks down) mRNA encoding a protein responsible for the cranial nerve disease. The polyionic complex of the present invention contains an antisense oligonucleotide including a DNA region as a nucleic acid, which may further encapsulate RNase H and may target and degrade mRNA or a nucleic acid encoding a protein responsible for the cranial nerve disease.

The documents cited in this specification are incorporated herein by reference in their entirety.

### Examples

### Materials

A guanidinylated polyethylene glycol-poly [(5-aminopentyl)-α,β-aspartamide] block copolymer (PEG-P (Asp-AP/G); PEG:Mn=2000, Mw/Mn=1.05, P (Asp-AP/G): DP 60, 80% guanidinylation) was synthesized [1]. A chemically modified antisense oligonucleotide (ASO) containing phosphorothioate (PS) backbone and a locked nucleic acid (LNA) was synthesized by Gene Design, Inc. (Osaka, Japan). The sequence of the ASO is: 5′-GGtcagctgccaatgcTAG-3′ (SEQ ID NO; 1) and targets the long noncoding RNA (lncRNA) of metastasis-associated lung adenocarcinoma transcript 1 (Malat1) (Malat1-ASO), Upper and lower case letters each denote a locked nucleic acid (LNA) (C denotes LNA methylcytidine) and DNA. Alexa Fluor™ 647 dye was attached to the 3′ end of the Malat1-ASO (ASO-AF647). Ribonuclease H (RNase H) with a molecular weight of 21 kDa derived from E. coli was purchased from Takara Bio Inc. (Shiga). Alexa Fluor 594 NHS Ester (AF594) was purchased from Thermo Fischer Scientific (Wilmington, DE, USA). Hepes (1 M, pH 7.3) was purchased from Amresco (Solon, OH, USA). BALB/c nude mice (female, 6 week-old) were purchased from Charles River Japan (Kanagawa, Japan). All animal experiments were performed according to the “Guidelines for the Care and Use of Laboratory Animals” of the University of Tokyo.

### Preparation of Polyionic Complex (PIC)

PEG-P (Asp-AP/G) was dissolved in 10 mM Hepes buffer at a concentration of 1 mg/mL and filtered using a 220 nm membrane filter. The polymer solution was mixed with a 15 μM ASO solution in 10 mM Hepes buffer at a predetermined charge ratio between the amino/guanidino groups of the PEG-P (Asp-AP/G) and the thiophosphate groups of the ASO, followed by vortex mixing at 2, 300 rpm for 2 minutes using a Mixmate (Eppendorf, Hamburg, Germany). The resulting vesicle composed of the ASO and cationic polymer is hereafter referred to as “ASOsome”.

### Electrophoretic Light Scattering/Dynamic Light Scattering (ELS/DLS)

Zeta potential, hydrodynamic diameter, and polydispersity index (PDI) of PIC samples were measured at 25° C. using a Zetasizer Nano-ZS instrument (Malvern Instruments, Worcestershire, UK) irradiated with a He—Ne ion laser (λ=633 nm). The zeta potential was calculated from the electrophoretic mobility by Smoluchowski's equation: (ζ=4 πην/ε (η: viscosity of Solvent, ν: electrophoretic mobility, ε: dielectric constant of solvent). The scattering angle was fixed at 173° for all measurements.

### Transmission Electron Microscopy (TEM)

PIC samples prepared at charge ratios of 1.4 and 2.4 were observed by TEM (JEM-1400; JEOL, Ltd., Tokyo, Japan) at a 120 kV. A copper plate was coated with collodion followed by carbon. A droplet of the sample solution was placed on top of the coated grid, and then a droplet of 50% ethanol solution containing 2 wt % uranyl acetate was placed on the surface of the grid with the sample to stain and dried at room temperature.

### Intranasal Administration

An inhalation mask that could be temporarily opened and closed was used for nasal administration [2]. BALB/c mice (female, 7 week-old) were fixed in a supine position and anesthetized using 2.5% isoflurane via the inhalation mask. Naked ASO or ASOsome (60 μM Malat1-ASO or ASO-AF6) was repeatedly administered using a micropipette into the left and right nostrils (2 ML dose, 30 seconds apart, 24 μL in total).

### Accumulation in Brain

At designated time points, mice were sacrificed, and the brain, olfactory bulb, and trigeminal nerve were removed. To compare the brain distribution of naked ASO or ASOsome, the brain including the olfactory bulb was sliced from left to right at 2 mm intervals using a brain slicer. The sliced specimens were observed with an in vivo imaging system (IVIS; PerkinElmer, Waltham, MA, USA) at Ex640/Em680 nm.

The olfactory bulb, trigeminal nerve, and brain were each homogenized with 1× lysis buffer (Promega, Madison, WI, USA), followed by quantitative analysis using a multibead shocker (Yasui Kikai, Osaka). Fluorescence intensity (FI) was measured using a Spark 20 M multimode microplate reader (Tecan, Maennedorf, Switzerland). Results were expressed as mean and s.d. (n=6).

To investigate the dynamics of positively charged ASOsome (ASOsome having a positive surface potential) from the nasal mucosa and trigeminal nerve to the brain, the olfactory bulb and trigeminal nerve were observed by IVIS to quantify FI of Ex640/Em680 nm at the same exposure time. Results were expressed as mean and s.d. (n=3).

To compare the naked ASO with the positively charged ASOsome, brain regions were isolated from each other, homogenized, and quantified with a Tecan microplate reader. Results were expressed as mean and s.d. (n=4).

### Preparation of RNase H-Encapsulated ASOsome

Preparation of RNase H labeled with AF594 (RNase H-AF594) and encapsulation in ASOsome having a positive surface potential by physical entrapment were performed according to a previous report [1]. RNase H-AF647 (2.5 mg/ml, 10 μL) was added to a prepared ASOsome solution (10 μM Malat1-ASO, 30 uL), followed by vortex mixing at 3000 rpm for 10 seconds. After 20 minutes, the prepared RNase H-encapsulated ASOsome solution was purified by ultrafiltration.

The amount encapsulated and encapsulation efficiency of ASO were calculated using a fluorospectrometer (Nanodrop 3300; Thermo Fischer Scientific), To the prepared ASO solution (10 μM Malat1-ASO), the designated concentration of RNase H-AF647 was added. After 20 minutes, centrifugation was performed at 20,000 g for 1 hour to measure the supernatant with a Nanodrop 3300.

The encapsulation of RNase H in the ASOsome was confirmed by analysis with fluorescence correlation spectroscopy (FCS), Autocorrelation curves of RNase H-AF594 and RNase H-encapsulated ASOsome were measured using LSM880-FCS mode with a sampling time of 10 seconds and a repeat time of 20 seconds.

The morphology of RNase H-encapsulated ASOsome was photographed by transmission electron microscopy (TEM). Droplets of sample solutions were placed on a plasma-treated copper plate, stained with 2 wt % uranyl acetate, and observed at 120 kV using JEM-1400 (JEOL, Tokyo, Japan).

### Gene Knockdown Assay

The gene silencing efficiency of RNase H-encapsulated ASOsome in the brain was evaluated by qRT-PCR. Naked ASO, empty ASOsome having a positive surface potential, a simple mixture of empty ASOsome and RNase H, and RNase H-encapsulated ASOsome were prepared so that the final concentration of Malat1-ASO was 60 μM. The N/P ratio of the RNase H-encapsulated ASOsome was the same in all cases. As a control, an RNase H-encapsulated ASOsome from a nonspecific sequence (scrambled) ASO was additionally prepared at the same concentration. All samples (24 μL, around 9.5 μg/mouse) were nasally administered to the BALB/c mice (female, 7 week-old) anesthetized with an inhalation mask. The mice were sacrificed, and brains were collected 48 hours after treatment. The cortex, striatum, and hippocampus were isolated from each brain, immediately weighed, and immersed in RNA protect Tissue Reagent (Qiagen, Valencia, CA, USA). RNA was extracted using RNeasy Mini Kit (Qiagen), CDNA was synthesized using ReverTra Ace (Toyobo, Osaka), and qRT-PCR was performed using ABI 7500 Fast Real-Time PCR System (Applied Biosystems, Foster City, CA, USA). Gapdh was used as a reference to determine the knockdown level of Malat1 lncRNA. The primer sequences were as follows:

Gapdh:
Forward
(SEQ ID NO: 2)
5′-TGT GTC CGT CGT GGA TCT GA-3′
and
Reverse
(SEQ ID NO: 3)
5′-TG CTG TTG AAG TCG CAG GAG-3′
Malat1:
Forward
(SEQ ID NO: 4)
5′-GAG CTC GCC AGG TTT ACA GT-3′
and
Reverse
(SEQ ID NO: 5)
5′-AAC TAC CAG CAA TTC CGC CA-3′

Results were expressed as mean and s.d. (n=4).

### Statistical Analysis

Comparison between groups was performed by one way ANOVA and post-hoc test. Differences were considered statistically significant when p<0.05,

### Results

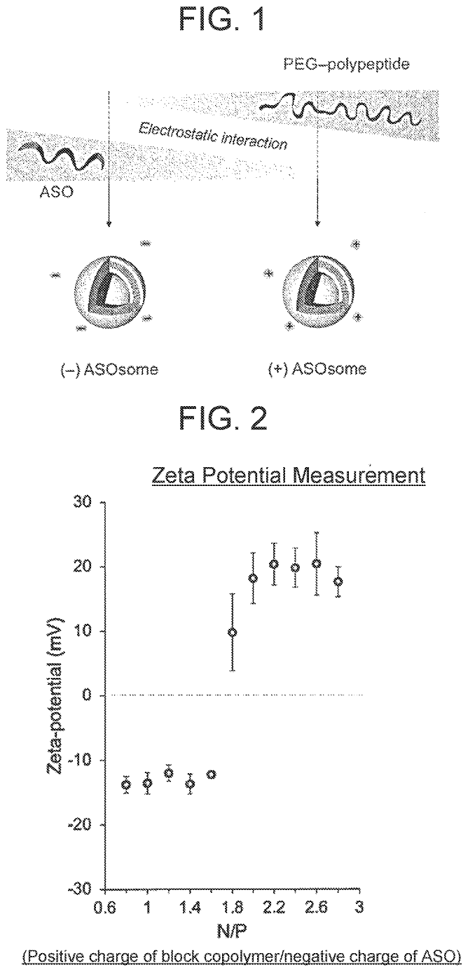

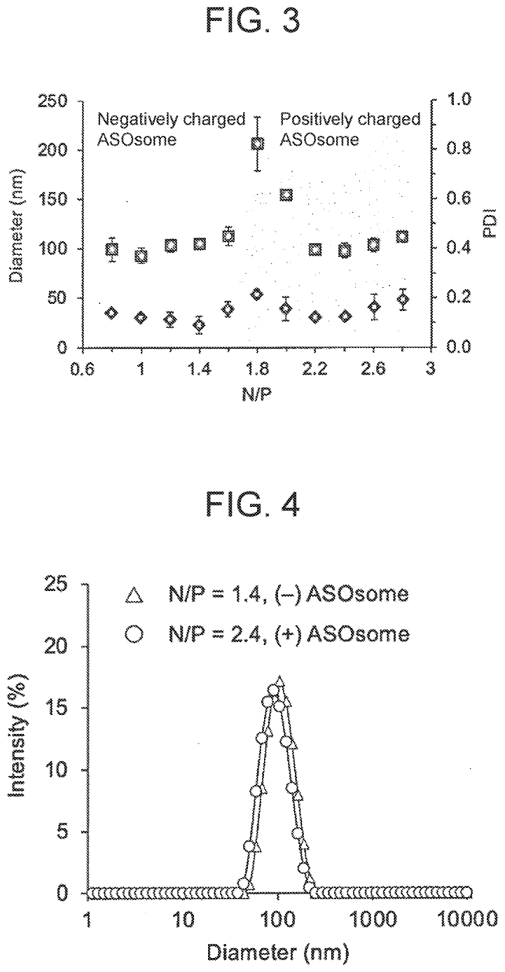

The polyionic complex is formed by complexing a cationic polymer and an anionic polymer by an ionic bond. In the present Example, a nucleic acid was used as the anionic polymer, and a polypeptide to which polyethylene glycol (PEG) was linked to increase biocompatibility was used as the cationic polymer. The nucleic acid used was an antisense oligonucleotide directed against the target gene Malat1. Varying the mixing ratio of the cationic polymer and the anionic polymer altered the zeta potential of the ASOsome (see FIGS. 1 and 2). The average particle size according to dynamic light scattering (DIS) was about 100 nm, and the polydispersity index (PDI) was 0.2 or lower, while the particle size was about 200 nm when the N/P ratio was about 1.8 (FIG. 3). The particle size distribution of ASOsome having an N/P ratio of 1.4 and ASOsome having an N/P ratio of 2.4 was as shown in FIG. 4. As shown in FIG. 4, the particle size distribution was independent of the zeta potential and independent of the N/P ratio.

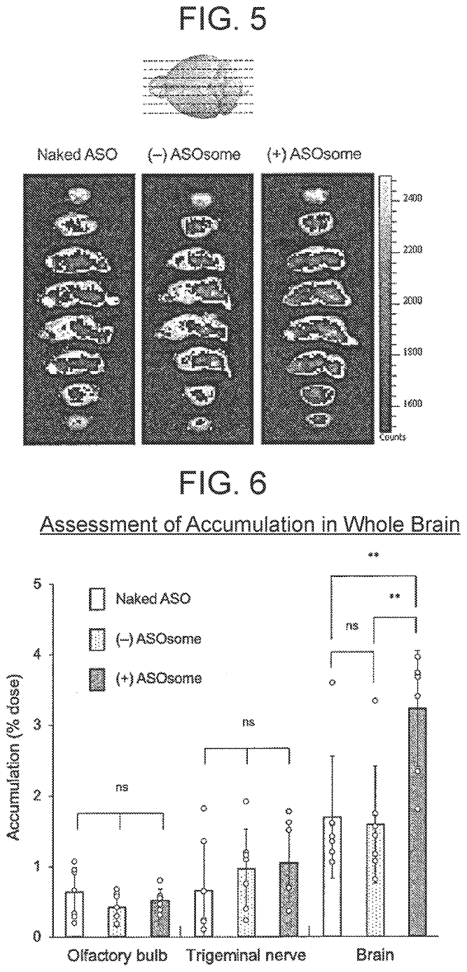

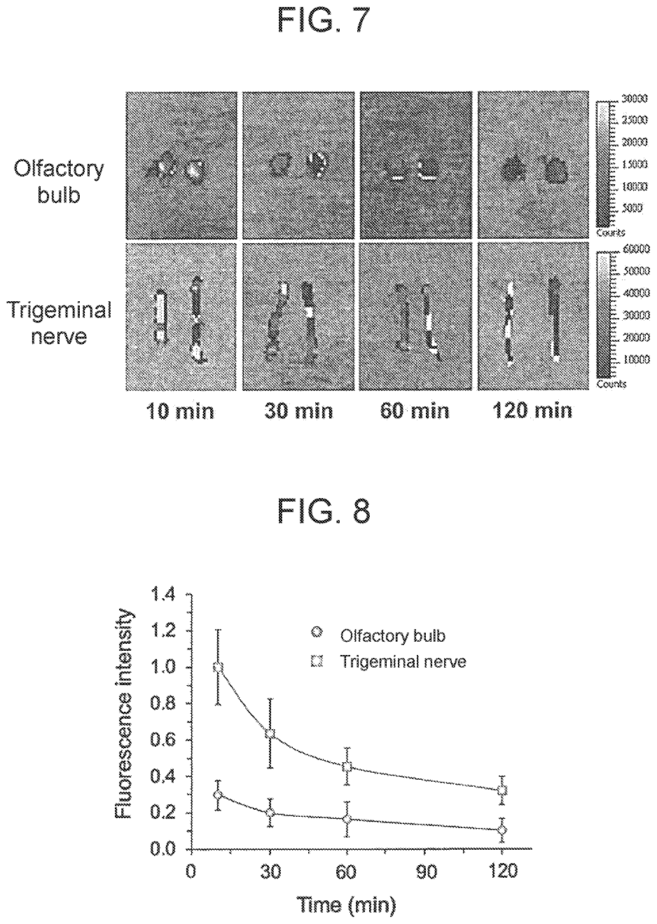

ASOsome was administered to the mice through the nasal mucosa. The ASOsome was shown to be able to deliver a nucleic acid to the brain, suggesting that the ASOsome may reach the brain by some route. The positively charged ASOsome was found to have higher delivery efficiency to the brain as compared with the negatively charged ASOsome (FIG. 5). The accumulation of ASOsome administered through the nasal mucosa in the olfactory bulb, trigeminal nerve, and brain was as shown in FIGS. 6 to 8. The results suggested that, besides the brain, the ASOsome also accumulated in the olfactory bulb and trigeminal nerve and that the ASOsome administered through the nasal mucosa migrated to the brain via the olfactory or trigeminal nerves (e.g., through axons). The results do not necessarily rule out the existence of a transepithelial pathway for the migration of ASOsome from the nasal mucosa to the cerebrospinal fluid across an epithelial cell layer, although the high accumulation of positively charged ASOsome suggests that the positive charge has an important technical significance in the transport pathway to the brain. What is important here is not only that ASOsome is delivered more efficiently than naked ASO, but also that it migrates to the brain to the same extent as ASO. Since a polymer assembly such as ASOsome migrates to brain tissue by nasal mucosal administration, a system for more stably delivering a nucleic acid or an inclusion to the brain has been established.

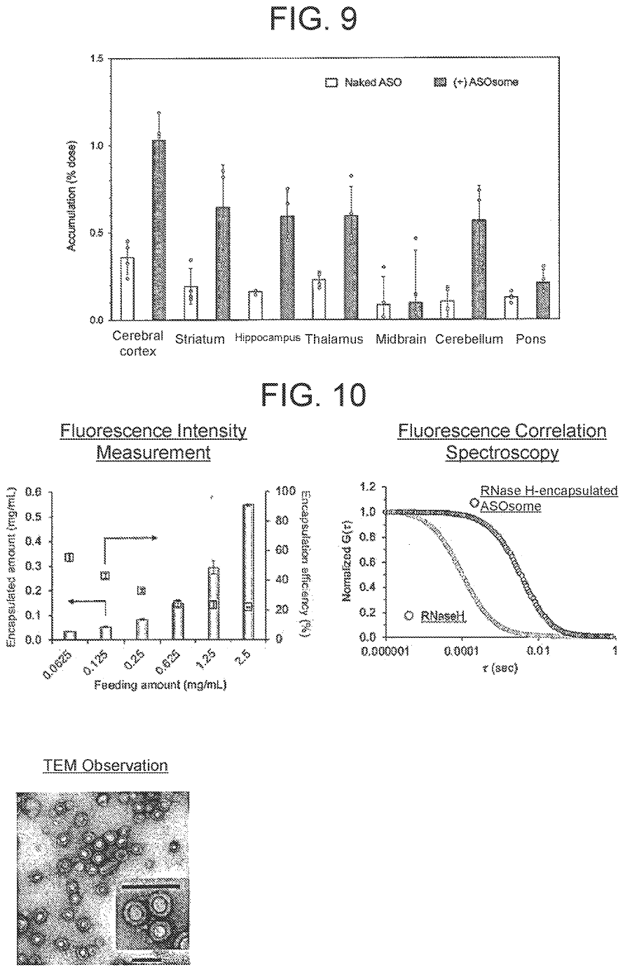

The distribution of the ASOsome administered through the nasal mucosa in the brain was confirmed. The results were as shown in FIG. 9. The ASOsome accumulated for all confirmed brain regions.

For the ASOsome, a DNA antisense oligonucleotide was used as an anionic polymer. The antisense oligonucleotide hybridizes with RNA to form a DNA-RNA double-stranded hybrid when reaching the cytoplasm. Since double-stranded nucleic acids of DNA and RNA are targeted for degradation by RNase H, an attempt was made to further impart a function of enzymatically degrading target molecules to ASOsome by encapsulating RNase H in the ASOsome. The RNase H-encapsulated ASOsome thus prepared was subjected to fluorescence intensity measurement, fluorescence correlation spectroscopy, and TEM observation. The results were as shown in FIG. 10. As shown in FIG. 10, the RNase H-encapsulated ASOsome behaved differently compared to the free RNase H, demonstrating that the RNase H was encapsulated in the ASOsome. In the TEM observation, a space was observed in a membrane, and the RNase H was presumably encapsulated in the space surrounded by the membrane.

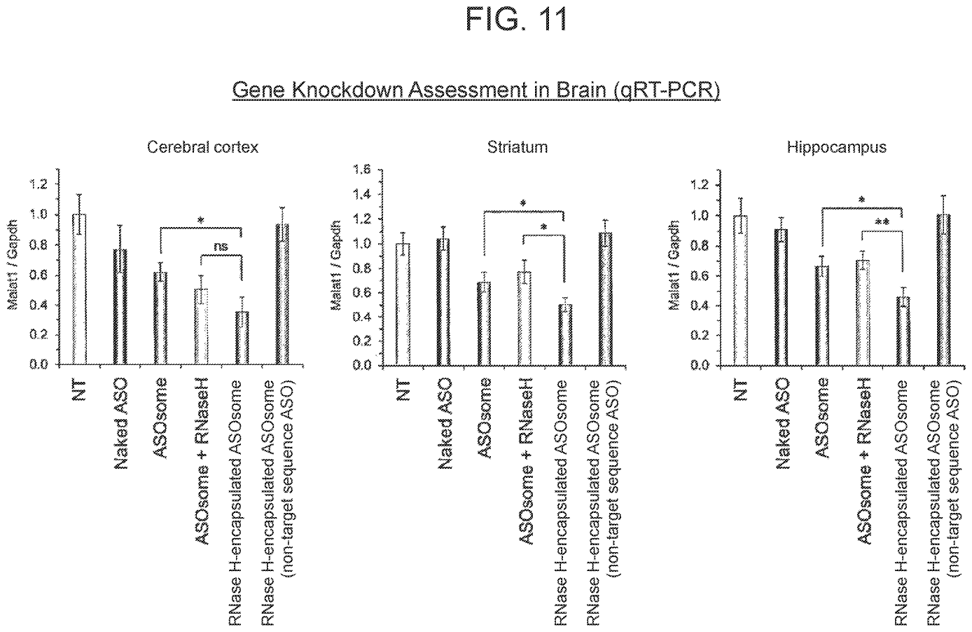

The obtained RNase H-encapsulated ASOsome was administered to a mouse through the nasal mucosa to confirm the knockdown efficiency of the target gene in the brain region. The results were as shown in FIG. 11. As shown in FIG. 11, the ASOsome effectively knocked down the target gene in each of the cerebral cortex, striatum, and hippocampus. The RNase H-encapsulated ASOsome also showed a greater knockdown effect as compared with the ASOsome-treated group and the groups in which RNase H and ASOsome were separately administered (see FIG. 11). Accordingly, the encapsulation of RNase H in the ASOsome served to enhance the action of ASOsome. The results that the ASOsome and the RNase H-encapsulated ASOsome were able to silence target genes in the brain suggest that the ASOsome was able to release intact nucleic acids and RNase H into the cytoplasm, at least after reaching the brain, indicating that the ASOsome was suitable for nucleic acid delivery into brain cells.

### REFERENCES

1. Noncovalent Stabilization of Vesicular Polyion Complexes with Chemically Modified/Single-Stranded Oligonucleotides and PEG-b-guanidinylated Polypeptides for Intracavity Encapsulation of Effector Enzymes Aimed at Cooperative Gene Knockdown. B. S. Kim, M. Naito, H, Chaya, M. Hori, K. Hayashi, H. S. Min, Y. Yi, H. J. Kim, T. Nagata, Y. Anraku, A. Kishimura, K. Kataoka, K. Miyata, Biomacromolecules, 2020, 21, 4365-4376.
2. Novel Methods for Intranasal Administration Under Inhalation Anesthesia to Evaluate Nose-to-Brain Drug Delivery. T. Kanazawa, M. Fukuda, N. Suzuki, T. J. Suzuki, J. Vis. Exp, 2018, 141, e58485.

1. A polyionic complex comprising:
(i) a block copolymer comprising (i-1) an uncharged hydrophilic polymer block and (i-2) a polymer block comprising a cationic amino acid;
(ii) a single-stranded nucleic acid that is an anionic polymer,
wherein the polyionic complex has a positive surface potential and is capable of delivering the nucleic acid from outside a cell to cytoplasm.

2. The polyionic complex according to claim 1, wherein an N/P ratio is 2.0 or more, where N denotes a valence of a positive charge in the block copolymer, and P denotes a valence of a negative charge in the nucleic acid.

3. The polyionic complex according to claim 1, wherein the nucleic acid is an antisense oligonucleotide directed against a target RNA.

4. The polyionic complex according to claim 1, wherein the nucleic acid is an antisense oligonucleotide directed against a target RNA, the antisense oligonucleotide consisting of DNA or comprising at least a contiguous nucleotide portion consisting of DNA, and the polyionic complex encapsulates RNase H, whereby a hybrid of the target RNA and the antisense oligonucleotide formed upon contact of the antisense oligonucleotide with the target RNA can be degraded.

5. The polyionic complex according to claim 1, wherein the uncharged hydrophilic polymer block is a polyalkylene glycol block.

6. A composition comprising the polyionic complex according to claim 1.

7. A method of administering the composition of claim 6, the method comprising nasally administering the composition according to claim 6 to nasal mucosa of a subject.

8. The method according to claim 7, wherein the method comprises delivering an effective amount of a nucleic acid to brain tissue of a subject.

9. The method according to claim 7, wherein the method comprises delivering an effective amount of a nucleic acid to trigeminal nerve or olfactory bulb of a subject.

10. The method according to any one of claims 7 to 9, wherein the composition comprises an antisense oligo DNA directed against a target gene, thereby suppressing expression of the target gene in brain tissue or trigeminal nerve cells of a subject.

11. The method according to claim 10, wherein the polyionic complex in the composition encapsulates RNase H.


SEQUENCE LISTING (5 sequences)

SEQ ID NO: 1 - DNA, 19 bp, synthetic construct
  misc_feature 1..19: note = Malat1-ASO
  source 1..19: mol_type = other DNA
ggtcagctgccaatgctag

SEQ ID NO: 2 - DNA, 20 bp, synthetic construct
  misc_feature 1..20: note = Gapdh forward primer
  source 1..20: mol_type = other DNA
tgtgtccgtcgtggatctga

SEQ ID NO: 3 - DNA, 20 bp, synthetic construct
  misc_feature 1..20: note = Gapdh reverse primer
  source 1..20: mol_type = other DNA
tgctgttgaagtcgcaggag

SEQ ID NO: 4 - DNA, 20 bp, synthetic construct
  misc_feature 1..20: note = Malat1 forward primer
  source 1..20: mol_type = other DNA
gagctcgccaggtttacagt

SEQ ID NO: 5 - DNA, 20 bp, synthetic construct
  misc_feature 1..20: note = Malat1 reverse primer
  source 1..20: mol_type = other DNA
aactaccagcaattccgcca